(19) United States
(12) Patent Application Publication
(10) Pub. No.: US 2026/0283604 A1
(43) Pub. Date: Sep. 24, 2026

(54) Adapter Devices, Systems, and Methods for Image Guided Surgery

(71) Applicant: Stryker Corporation, Portage, MI (US)

(72) Inventors: Vinunti Trivedi, London (GB); Colin Dowling, Cork (IE); Alexander Houck, Minnetonka, MN (US); Cameron Erber, New York, NY (US); Anthony Hanson, Chaska, MN (US); Daniel O'Brien, Cork (IE)

(21) Appl. No.: 19/572,963

(22) Filed: Mar. 20, 2026

Related U.S. Application Data
(60) Provisional application No. 63/775,798, filed on Mar. 21, 2025.

(51) Int. Cl. A61B 17/00; A61B 34/20

(52) U.S. Cl. CPC A61B 17/00; A61B 34/20; A61B 2017/00486; A61B 2034/2051

(57) ABSTRACT

In an example, an adapter for coupling an image guidance sensor to an instrument includes a clamp frame, a screw, an upper jaw, a lower jaw, and a sensor. The clamp frame includes an upper portion, a lower portion, and a bridge portion between the upper and lower portions. The clamp frame defines a frame cavity between the upper portion and the lower portion. The screw is threadably coupled to the upper portion. The upper jaw is disposed in the frame cavity and coupled to the screw. The upper jaw and the lower jaw define a jaw space. The frame cavity provides access to the jaw space. The screw is configured to move upper jaw relative to the lower jaw to adjust a size of the jaw space. The sensor is configured to generate a signal that is indicative of a position of the sensor responsive to an electromagnetic field.

### CROSS-REFERENCE TO RELATED APPLICATIONS

The present application claims the benefit of U.S. Provisional Application No. 63/775,798, filed Mar. 21, 2025, the contents of which are hereby incorporated by reference in their entirety.

### FIELD

The present disclosure relates to image guided surgery and, in particular, to adapter devices, systems, and methods for image guided surgery.

### BACKGROUND

Medical imaging techniques (e.g., computerized tomography (CT) and magnetic resonance image (MRI)) allow medical personal to view three-dimensional images of the organs and tissue within a person. In addition to medical imaging being useful for diagnosing a patient's condition, medical imaging can be used as an aid in performing surgical procedures. Specifically, medical imaging can be used to perform what is referred to as image guided surgery.

In general, image guided surgery can include obtaining one or more preoperative images of an anatomical region in which the surgical procedure will be performed. Image guided surgery further includes registering the preoperative image(s) to the patient by identifying and matching specific anatomical landmarks on the patient's body to the same points on the preoperative images. A surgical instrument can be provided with a sensor that can generate position data responsive to an electromagnetic field, and communicate the position data to a navigation system. The sensor, integrated or attached to the surgical instrument, detects the electromagnetic field created by a field generator and communicates its position and orientation data to the navigation system in real-time. In this way, a surgeon can use the surgical instrument as usual while the sensor provides real-time position data, which the navigation system can display on the screen to provide an indication of a location and/or an orientation of the surgical instrument within the body of the patient (e.g., by displaying a position of the surgical instrument overlaid on the preoperative images). Thus, the surgeon performing the procedure is able to see where, in the body, the instrument is located even though the surgical site is not directly exposed to the eye.

Image guided surgery can help to increase accuracy and precision, which can lead to better surgical results and/or reduced complications. Additionally, image guided surgery can help surgeons to perform complex procedures more efficiently, which can help to reduce procedure time and improve post-surgery recovery.

### BRIEF DESCRIPTION OF THE FIGURES

The novel features believed characteristic of the illustrative embodiments are set forth in the appended claims. The illustrative embodiments, however, as well as a preferred mode of use, further objectives and descriptions thereof, will best be understood by reference to the following detailed description of an illustrative implementation of the present disclosure when read in conjunction with the accompanying figures, wherein:

FIG. 1 depicts a simplified block diagram of an adapter for coupling an electromagnetic guidance sensor to a surgical instrument, according to an example.

FIG. 2A depicts a perspective view of an implementation of the adapter shown in FIG. 1, according to an example.

FIG. 2B depicts the adapter of FIG. 2A coupled to a surgical instrument, according to the example.

FIG. 2C depicts a perspective view of a screw, according to an example.

FIG. 2D depicts a cross-sectional view of an upper jaw for coupling with the screw shown in FIG. 2C, according to an example.

FIG. 2E depicts a cross-sectional view of the adapter shown in FIG. 2A including the screw of FIG. 2C coupled to the upper jaw of FIG. 2D, according to the example.

FIG. 3A depicts an adapter with an upper jaw in the first position, according to an example.

FIG. 3B depicts the adapter shown in FIG. 3A with the upper jaw in a second position, according to an example.

FIG. 4 depicts a thumbscrew head of an adapter, according to an example.

FIG. 5 depicts an upper jaw and a lower jaw of an adapter, according to an example.

FIG. 6A depicts a perspective view of an assembly of the screw, the thumbscrew head, and a torque limiter, according to an example.

FIG. 6B depicts a bottom view of the assembly shown in FIG. 6A, according to an example.

FIG. 6C depicts a cross-sectional view of the assembly shown in FIG. 6A, according to an example.

FIG. 7 depicts a cross-sectional view of an assembly of the screw, the thumbscrew head, and a torque limiter, according to another example.

FIG. 8 depicts a nut of an adapter, according to an example.

FIG. 9A depicts an adapter, according to an example.

FIG. 9B depicts a lower jaw for the adapter shown in FIG. 9A, according to the example

FIG. 9C depicts a first view of a clamp frame for the adapter shown in FIG. 9A, according to an example.

FIG. 9D depicts a second view the clamp frame for the adapter shown in FIG. 9A, according to the example.

FIG. 10 depicts a simplified block diagram of an image guidance system, according to an example.

FIG. 11 depicts a flowchart for a process of coupling an adapter including an image guidance sensor to a surgical instrument, according to an example.

FIG. 12 depicts a flowchart for a process of making an adapter including an image guidance sensor to a surgical instrument, according to an example.

### DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As noted above, image guided surgery can provide a number of benefits when performing certain surgical procedures in a body of a patient. However, not all surgical instruments have an integrated sensor for image guided surgery. For example, some surgical instruments were not originally designed with integrated sensor, and/or it may be cost prohibitive to incorporate a sensor into all surgical instruments that may be used during image guided surgical procedures.

To use such surgical instruments in an image guided surgical procedure, an adapter including the sensor can be coupled to the surgical instrument. Some existing adapters for image guided surgery include a clamp that is configured to clamp onto a shaft of a surgical instrument. In some implementations, the existing adapters may include a screw mechanism to couple and decouple the adapter to the shaft of the surgical instrument. For instance, turning the screw causes a jaw of the clamp to move up or down to increase or decrease an amount of space within the clamp.

One challenge associated with image guided surgery adapters that include a screw mechanism is that it can be difficult to know when the clamp has been suitable tightened onto the shaft of the surgical instrument. If the clamp is too loose, the adapter may inadvertently rotate on the shaft. On the other hand, if the clamp applies too much force to the shaft, the shaft may be damaged by the clamp.

Another challenge associated with image guided surgery adapters is that one or more jaws of the clamp may deflect to an off-axis orientation due a force between the jaw(s) and the surgical instrument. This deflection of the jaw(s) can reduce a holding force of the clamp, which may lead to inadvertent rotation of the adapter on the shaft of the surgical instrument.

Within examples, the present disclosure provides for adapters, methods of using adapters, and methods of manufacturing adapters that can address one or more of these and other challenges associated with image guided surgery adapters.

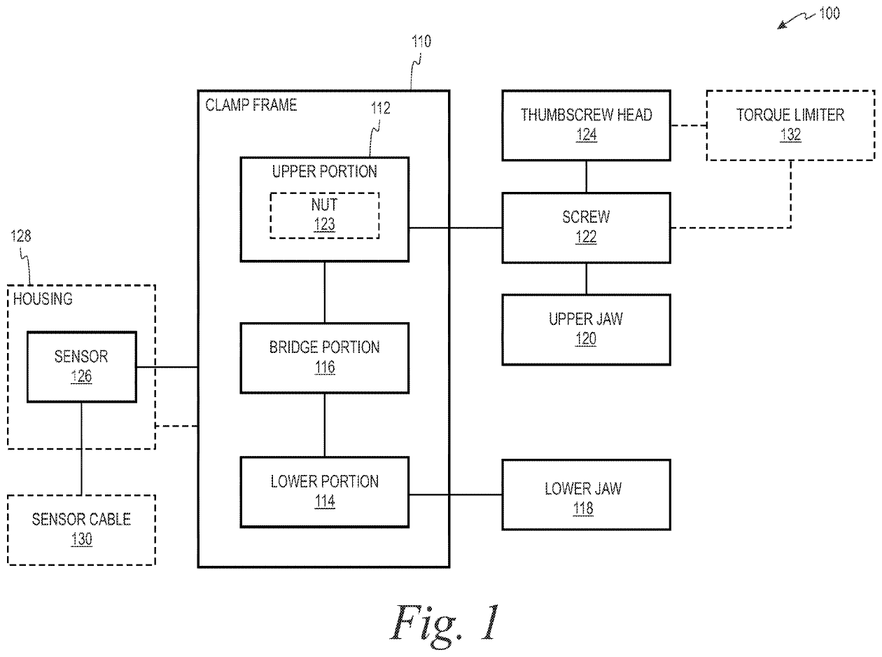

Referring to FIG. 1, a simplified block diagram of an adapter 100 for coupling an electromagnetic guidance sensor to a surgical instrument is shown according to an example. As shown in FIG. 1, the adapter 100 includes a clamp frame 110. The clamp frame 110 includes an upper portion 112, a lower portion 114, and a bridge portion 116 between the upper portion 112 and the lower portion 114. The clamp frame 110 defines a frame cavity between the upper portion 112 and the lower portion 114. As described in further detail below, the frame cavity of the clamp frame 110 can provide a space for receiving the surgical instrument between the upper portion 112 and the lower portion 114 of the clamp frame 110. For example, the clamp frame 110 can have a C-shape in which the upper portion 112 extends in a first plane, the lower portion 114 extends in a second plane that can be parallel to the first plane, and the bridge portion 116 can extend in a third plane that can be orthogonal to the first plane and the second plane. In this arrangement, the C-shape of the clamp frame 110 can define the frame cavity in which the surgical instrument can be positioned.

Additionally, as shown in FIG. 1, the adapter 100 includes a lower jaw 118 and an upper jaw 120. In some implementations, the lower jaw 118 and the lower portion 114 of the clamp frame 110 can be formed a single, monolithic structure. In such examples, the lower portion 114 of the clamp frame 110 can define the lower jaw 118. This can help to enhance structural strength and durability and/or simplify manufacturing. In other implementations, the lower jaw 118 can be fixedly coupled to the lower portion 114 of the clamp frame 110. This can help provide for adjustability (e.g., in implementations in which different types of the lower jaw 118 can be interchangeably coupled to the clamp frame 110) and/or provide manufacturing the clamp frame 110 and the lower jaw 118 from different materials and/or using different manufacturing processes.

As shown in FIG. 1, the adapter 100 also includes a screw 122 that is threadably coupled to the upper portion 112 of the clamp frame 110. The upper jaw 120 is disposed in the frame cavity of the clamp frame 110 and coupled to the screw 122. In this arrangement, the upper jaw 120 and the lower jaw 118 define a jaw space for receiving the surgical instrument between the upper jaw 120 and the lower jaw 118. As such, the jaw space can be a portion of the frame cavity that is between the upper jaw 120 and the lower jaw 118.

The screw 122 is configured to move upper jaw 120 relative to the lower jaw 118 to adjust a size of the jaw space between the upper jaw 120 and the lower jaw 118. For example, the upper jaw 120 can be moved in a direction toward the upper portion 112 of the clamp frame 110 and away from the lower jaw 118 to increase the size of the jaw space, and the upper jaw 120 can be moved in a direction toward the lower portion 114 and toward the lower jaw 118 to reduce the size of the jaw space. This can provide for increasing the jaw space for inserting the surgical instrument into the jaw space and removing the surgical instrument from the jaw space, and provide for decreasing the jaw space to couple (e.g., clamp) the adapter 100 to the surgical instrument.

Within examples, the screw 122 and the upper portion 112 of the clamp frame 110 can be configured to transduce rotational movement of the screw 122 into axial movement of the screw 122 along a longitudinal axis of the screw 122. For example, the screw 122 can have a lower end that is coupled to the upper jaw 120, an upper end that extends above the upper portion 112 of the clamp frame 110, and a screw shaft that extends through the upper portion 112 of the clamp frame 110. The screw shaft of the screw 122 can include an external thread that threadedly engages an internal thread of the upper portion 112 of the clamp frame 110. In some implementations, the internal thread can be integrally formed with the upper portion 112 of the clamp frame as a single monolithic structure. In other implementations, the internal thread can be provided by a nut 123 that is coupled to the upper portion 112 of the clamp frame 110.

Within examples, the lower end of the screw 122 can be rotatably coupled to the upper jaw 120. This can allow the upper jaw 120 to remain rotationally fixed relative to the surgical instrument in the jaw space while the screw 122 is rotated to tighten the upper jaw 120 against the surgical instrument.

As shown in FIG. 1, the adapter 100 can include a thumbscrew head 124 coupled to the upper end of the screw 122. The thumbscrew head 124 can be rotatable relative to the clamp frame 110 to rotate the screw 122 relative to the upper portion 112, and axially move the screw 122 and the upper jaw 120 relative to the clamp frame 110 and the lower jaw 118 (e.g., along the longitudinal axis of the screw 122). In this way, a user can grip and rotate the thumbscrew head 124 to actuate the screw 122 and move the upper jaw 120 relative to the lower jaw 118, as described above. In general, the thumbscrew head 124 can extend laterally from the screw body to increase a surface area for gripping the screw 122 at the thumbscrew head 124.

In some examples, the thumbscrew head 124 can be fixedly and non-rotatably coupled to the upper end of the screw 122 such that the thumbscrew head 124. In other examples, the adapter can include a torque limiter 132 that is configured to couple the thumbscrew head 14 to the screw 122. As described in further detail below, the torque limiter 132 can be configured to rotationally couple the thumbscrew head 124 to the screw 122 when a torque applied to the thumbscrew head 124 in a first rotational direction is less than a predetermined threshold amount of torque, and the torque limiter 132 can be configured to rotationally decouple the thumbscrew head 124 from the screw 122 when the torque applied to the thumbscrew head 124 in the first rotational direction is greater than the predetermined threshold amount of torque.

In this arrangement, the user can rotate the thumbscrew head 124 to tighten the upper jaw 120 and the lower jaw 118 on the surgical instrument until the torque limiter 132 ceases to rotating the screw 122. As such, the user can consistently and reliably apply a predetermined amount of force to the surgical instrument in the jaw space between the upper jaw 120 and the lower jaw 118. The predetermined amount of force can be an amount that is suitable for securely coupling the adapter 100 to the surgical instrument without damaging the surgical instrument and while mitigating inadvertent movement (e.g., sliding or rotation) of the adapter 200 relative to the surgical instrument.

The torque limiter 132 can be further configured to rotationally couple the thumbscrew head 124 to the screw 122 when any torque is applied to the thumbscrew head 124 in a second rotational direction. This can allow the thumbscrew head 124 to always be able to move the upper jaw 120 away from the lower jaw 118 to decouple the adapter 100 from the surgical instrument. Indeed, the torque limiter 132 can be configured such that (i) rotating the thumbscrew head 124 in the first rotational direction with a torque that is less than the predetermined threshold amount of torque causes the upper jaw 120 to move towards the lower jaw 118, and (ii) rotating the thumbscrew head 124 in the second rotational direction causes the upper jaw 120 to move away from the lower jaw 118.

Additionally, as shown in FIG. 1, the adapter 100 includes an image guidance sensor 126 that is configured to generate a signal that is indicative of a position of the image guidance sensor 126 responsive to an electromagnetic field generated by a field generator. In general, the image guidance sensor 126 is coupled to the clamp frame 110. In some implementations, the image guidance sensor 126 can be disposed an internal cavity of the clamp frame 110. In such implementations, the clamp frame 110 can be formed from a material that does not cause interference problems for the image guidance sensor 126. For instance, the clamp frame 110 can be formed from a material that is non-magnetic and non-conductive, but has suitable strength and rigidity to withstand forces applied the clamp frame 110 when coupling the adapter 100 to the surgical instrument. As examples, the clamp frame 110 can be formed from titanium and/or Polyether ether ketone (PEEK).

In other implementations, the image guidance sensor 126 can be coupled to an exterior of the clamp frame 110 and enclosed by a housing 128 that is also coupled to the clamp frame 110 around the image guidance sensor 126. This can allow the clamp frame 110 to be formed from some materials that may not be suitable if the image guidance sensor 126 was disposed in an internal cavity of the clamp frame 110. The housing 128 can help to protect the image guidance sensor 126 enclosed in a spaced defined by the housing 128 and/or the clamp frame 110.

As described above, the image guidance sensor 126 can sense the electromagnetic field generated by the field generator at a surgical site, and, based on the electromagnetic field sensed by the image guidance sensor 126, generate the signal that is indicative of the position of the image guidance sensor 126. In one example, the image guidance sensor 126 can include a sensor coil (e.g., a wire of conductive material in a coil shape). In other examples, the image guidance sensor 126 can additionally or alternatively include a Hall Effect sensor, a magnet resistive sensor, and/or a fluxgate magnetometer.

As shown in FIG. 1, the image guidance sensor 126 can be coupled to a sensor cable 130. The sensor cable 130 can be configured to transmit the signal from the image guidance sensor 126 to a controller of an image guided surgery system. An example image guided surgery system will be described in further detail below.

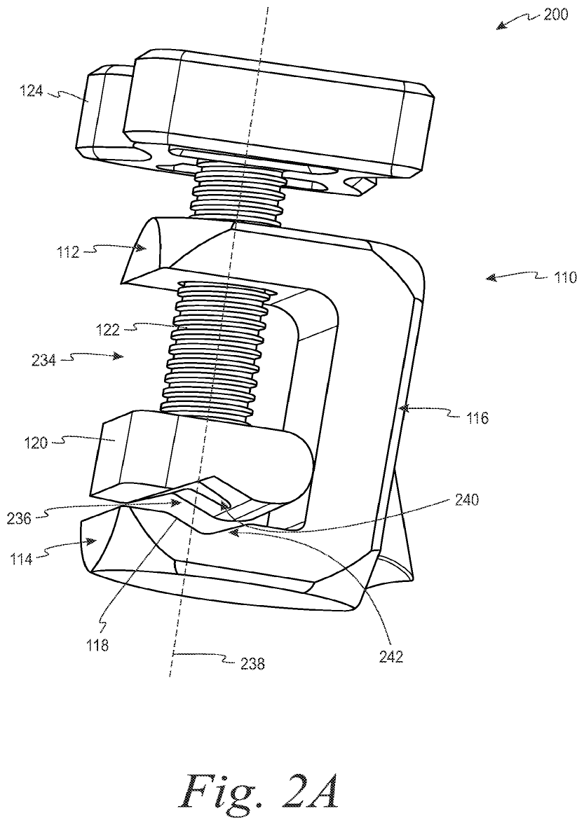

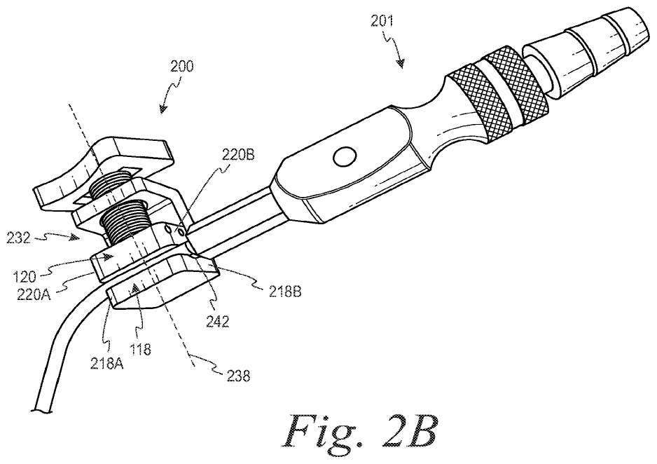

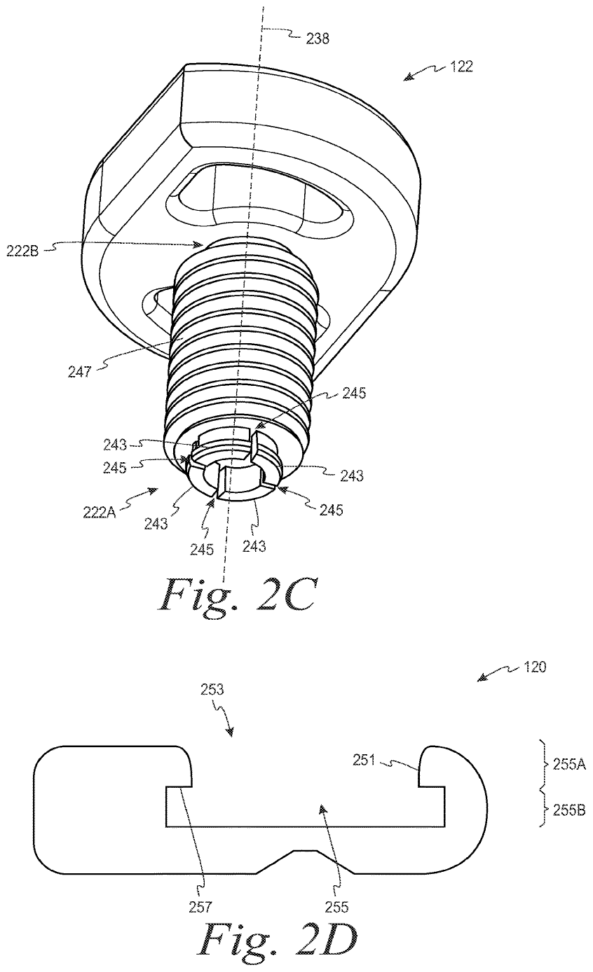

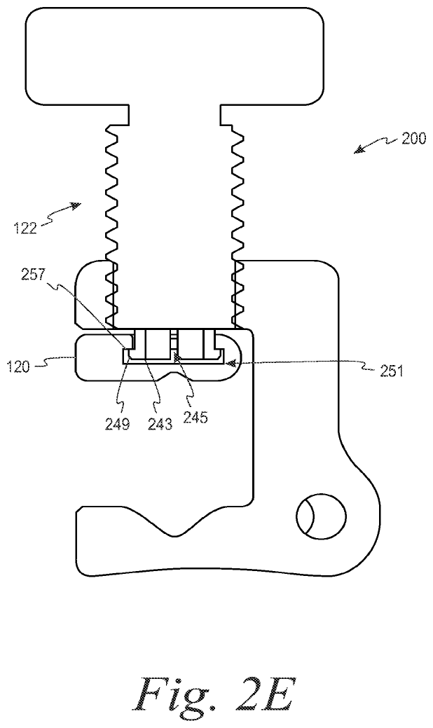

FIGS. 2A-2B depict an adapter 200 as an implementation of the adapter 100 shown and described with respect to FIG. 1, according to an example. In particular, FIG. 2A depicts a perspective view of the adapter 200, and FIG. 2B depicts the adapter 200 coupled to a surgical instrument 201, according to the example.

As shown in FIG. 2A, the adapter 200 includes the clamp frame 110. The clamp frame 110 includes the upper portion 112, the lower portion 114, and the bridge portion 116 between the upper portion 112 and the lower portion 114. As shown in FIG. 2A, the upper portion 112, the lower portion 114, and the bridge portion 116 are formed as a single, monolithic structure. This can help to enhance a structural strength and durability of the clamp frame 110, and/or simplify manufacturing of the clamp frame 110.

Additionally, as shown in FIG. 2A, the adapter 200 includes the lower jaw 118 and the upper jaw 120. In FIG. 2A, the lower jaw 118 and the lower portion 114 of the clamp frame 110 are formed a single, monolithic structure such that the lower portion 114 of the clamp frame 110 defines the lower jaw 118. However, in other implementations, the lower jaw 118 can be a separate component that is fixedly coupled to the lower portion 114 of the clamp frame 110.

As shown in FIG. 2A, the adapter 200 also includes the screw 122 that is threadably coupled to the upper portion 112 of the clamp frame 110. The upper end of the screw 122 is coupled to the thumbscrew head 124, the lower end of the screw 122 is rotatably coupled to the upper jaw 120, and the body of the screw is threadedly coupled to the upper portion 112 of the clamp frame 110. As described above, the lower end of the screw 122 can be rotatably coupled to the upper jaw 120 to allow the upper jaw 120 to remain rotationally fixed relative to the surgical instrument in the jaw space and the lower jaw 118 while the screw 122 is rotated to tighten the upper jaw 120 against the surgical instrument. Additionally, to help maintain the upper jaw 120 rotationally aligned with the lower jaw 118, the upper jaw 120 can be configured to abut against the bridge portion 116 of the clamp frame 110. In this arrangement, the upper jaw 120 can be rotationally aligned with the lower jaw 118 as the screw 122 moves the upper jaw 120 towards the lower jaw 118 and away from the lower jaw 118.

The clamp frame 110 further defines a frame cavity 234 between the upper portion 112 and the lower portion 114. As shown in FIGS. 2A-2B, the frame cavity 234 of the clamp frame 110 provides a space for receiving the surgical instrument 201 and moving the upper jaw 120 between the upper portion 112 and the lower portion 114 of the clamp frame 110. For instance, in FIGS. 2A-2B, the clamp frame 110 has a C-shape in which the upper portion 112 extends in a first plane, the lower portion 114 extends in a second plane that can be parallel to the first plane, and the bridge portion 116 can extend in a third plane that can be orthogonal to the first plane and the second plane. In this arrangement, the clamp frame 110 can define the frame cavity 234 in which the surgical instrument 201 can be positioned.

As shown in FIG. 2A, the upper jaw 120 is disposed in the frame cavity 234 of the clamp frame 110 and coupled to the screw 122. In this arrangement, the upper jaw 120 and the lower jaw 118 define a jaw space 236 for receiving the surgical instrument 201 between the upper jaw 120 and the lower jaw 118. As such, the jaw space 236 can be a portion of the frame cavity 234 that is between the upper jaw 120 and the lower jaw 118.

In FIGS. 2A-2B, the screw 122 has a longitudinal axis 238 extending between the upper end of the screw 122 and a lower end of the screw 122 (e.g., between the thumbscrew head 124 and the upper jaw 120), and the longitudinal axis 238 of the screw 122 extends through the jaw space 236 defined by the upper jaw 120 and the lower jaw 118. This can decrease a risk of deflection of the upper jaw 120 when the surgical instrument 201 is in the jaw space 236 and a force is applied to the upper jaw 120 by the screw 122. As a result, the arrangement of the screw 122, the upper jaw 120, and the lower jaw 118 can address some problems associated with some adapters in which the screw 122 is offset laterally from the jaw space 236 (e.g., problems of undesirable deflection of the upper jaw 120 as described above).

As shown in FIG. 2B, the longitudinal axis 238 of the screw 122 can further extend through the surgical instrument 201 when the surgical instrument 201 is positioned in the jaw space 236 between the upper jaw 120 and the lower jaw 118. This can further help to increase a holding force applied by the upper jaw 120 and the lower jaw 118 to the surgical instrument 201.

As shown in FIGS. 2A-2B, the upper jaw 120 can include a first recess 240 and the lower jaw 118 can include a second recess 242 that is opposite the first recess 240 of the upper jaw 120. The first recess 240 has a center axis between a first lateral side 220A of the upper jaw 120 and a second lateral side 220B of the upper jaw 120, and the second recess 242 has a center axis between a first lateral side 218A of the lower jaw 118 and a second lateral side 218B of the lower jaw 118. The center axis of the first recess 240 is parallel to the center axis of the second recess 242. In this arrangement, the first recess 240 of the upper jaw 120 and the second recess 242 of the lower jaw 118 can help to improve surface contact with the surgical instrument 201.

As shown in FIGS. 2A-2B, the center axis of the first recess 240 and the center axis of the second recess 242 can be orthogonal to a longitudinal axis 238 of the screw 122 (and the third plane of the bridge portion 116, which can be parallel to the longitudinal axis 238 of the screw 122). This can allow the upper jaw 120 and the lower jaw 118 to clamp onto the surgical instrument 201 such that a longitudinal axis of a portion of the surgical instrument 201 in the jaw space 236 (e.g., a shaft of the surgical instrument 201) is orthogonal to the longitudinal axis 238 of screw 122 and, thus, orthogonal to a direction of the force applied to the surgical instrument 201 by the upper jaw 120 and the lower jaw 118.

In some examples, a shape of the first recess 240 can mirror a shape of the second recess 242. This can help the upper jaw 120 and the lower jaw 118 to more equally distribute the force applied to the surgical instrument 201 between the upper jaw 120 and the lower jaw 118 in instances in which the portion of the surgical instrument 201 that is in the jaw space 236 has a symmetrical cross-section (e.g., a circular shaft). However, in other examples, the first recess 240 and the second recess 242 can have different shapes. This may be beneficial in some implementations in which the surgical instrument has a non-circular and/or asymmetric cross-section and it is desirable to provide the upper jaw 120 and/or the lower jaw 118 with respective shapes that better fit different portions of the cross-section of the surgical instrument 201.

For instance, in other implementations, one of the lower jaw 118 can have the second recess 242 and the upper jaw 120 can omit the first recess 240, or vice versa. This may be beneficial in some implementations in which the surgical instrument 201 has a pentagon shaped cross-section.

However, in the implementation shown in FIGS. 2A-2B, the lower jaw 118 and the upper jaw 120 can be configured to have the second recess 242 and the first recess 240, respectively, that can couple to a pentagon shaped cross-section of the surgical instrument 201. For instance, as shown in FIG. 2A, the lower jaw 118 and the upper jaw 120 include planar portions on opposing sides of the first recess 240 and the second recess 242. In this arrangement, a corner of the pentagon shaped cross-section can be received in the first recess 240 or the second recess 242, and a flat portion of the pentagon shaped cross-section (which is opposite the corner received in the first recess 240 or the second recess 242) can engage the planar portions of the lower jaw 118 or the upper jaw 120.

In some examples, the first recess 240 of the upper jaw 120 can be smaller than the second recess 242 of the lower jaw 118 to help increase a size of the planar portions of the upper jaw 120, or vice versa. This can help to increase a surface of the planar portion while retaining both the first recess 240 and the second recess 242, which can improve coupling of the adapter 100 to the pentagon shaped cross-section of the surgical instrument 201 as well as differently shaped surgical instruments 201.

In FIG. 2A, the first recess 240 and the second recess 242 each have a triangular shape. This can, for instance, provide for at least four points of contact with the surgical instrument 201 in some implementations. In other examples, the first recess 240 and/or the second recess 242 can have other shapes, which may provide for improved surface contact with certain types of the surgical instrument 201. For instance, in another example, the first recess 240 and/or the second recess 242 can have a flat surface profile.

In some examples, the clamp frame 110, the upper jaw 120, the lower jaw 118, and the screw 122 can each consist of a metal material (e.g., titanium such as titanium grade 5). In other examples, the clamp frame 110, the upper jaw 120, the lower jaw 118, and/or the screw 122 be formed from one or more materials selected from a group consisting of: stainless steel and PEEK.

As described above, the lower end of the screw 122 can be rotatably coupled to the upper jaw 120. This can allow the upper jaw 120 to remain rotationally fixed relative to the surgical instrument in the jaw space while the screw 122 is rotated to tighten the upper jaw 120 against the surgical instrument.

FIGS. 2C-2E depict an implementation of a coupling arrangement for the screw 122 and the upper jaw 120, according to one example. In particular, FIG. 2C depicts a perspective view of the screw 122, FIG. 2D depicts a cross-sectional view of the upper jaw 120, and FIG. 2E depicts a cross-sectional view of the adapter 200 including the screw 122 of FIG. 2C coupled to the upper jaw 120 of FIG. 2D, according to the example.

As shown in FIG. 2C, the screw 122 includes a lower end 222A and an upper end 222B. Additionally, the screw 122 has a longitudinal axis 238 extending between the upper end 222B of the screw 122 and the lower end 222A of the screw 122A. In this example, the lower end 222A of the screw 122 can include a plurality of resilient fingers 243 separated by a plurality of axial slots 245, allowing the resilient fingers 243 to flex radially inwardly towards the longitudinal axis 238. Within examples, the resilient fingers 243 can be biased towards a rest position such that, after being radially flexed inwards toward the longitudinal axis 238, the resilient fingers 243 return to the rest position shown in FIG. 2C.

In some examples, the axial slots 245 can extend from the lower end 222A of the screw 122 to a point at or below a threaded section 247 of the screw 122. This can help to mitigate issues that may result if the axial slots 245 extended into the threaded section 247.

As shown in FIG. 2E, each of the resilient fingers 243 can include a respective flange 249 that extends outwardly from the resilient finger 243 (e.g., in a direction extending away from and orthogonal to the longitudinal axis 238). As explained in further detail below, the flanges 249 can help to axial retain the lower end 222A of the screw 122 in the upper jaw 120.

As shown in FIG. 2D, the upper jaw 120 can include a receptacle 251 that is configured to receive and axially retain the lower end 22A of the screw 122. The receptacle 251 can include an aperture 253 at an upper surface and an internal cavity 255 extending downwardly from the aperture 253. The internal cavity 255 can include an upper cavity section 255A and a lower cavity section 255B. The upper cavity section 255A can extend from the aperture 253 to the lower cavity section 255B. The lower cavity section 255B can extend from the upper cavity section 255A to a point that is above a lower surface of the upper jaw 120.

Additionally, as shown in FIG. 2D, the aperture 253 and the upper cavity section 255A can have a first diameter, the lower cavity section 255B can have a second diameter, and the second diameter can be greater than the first diameter. In this arrangement, the receptacle 251 can include a shoulder 257 at an interface between the upper cavity section 255A and the lower cavity section 255B. The shoulder 257 can assist in axially retaining the lower end 222A of the screw 122.

As shown in FIG. 2E, the first diameter of the aperture 253 and the upper cavity section 255A can be less than a diameter defined by the flanges 249 of the lower end 222A of the screw 122 when the resilient fingers 243 are in the rest position. Also shown in FIG. 2E, the second diameter of the lower cavity section 255B can be greater than the diameter defined by the flanges 249.

In this arrangement, the lower end 22A of the screw 122 can be coupled to the upper jaw 120 by flexing the resilient fingers 243 inwardly from the rest position towards the longitudinal axis 238 such that the diameter of the lower end 222A at the flanges 249 is less than the first diameter of the aperture 253 and the upper cavity section 255A. This allows the flanges 249 to be inserted through the aperture 253 and the upper cavity section 255A. When the flanges 249 pass from the upper cavity section 255A to the lower cavity section 255B, the resilient fingers 243 automatically move back towards the rest position as shown in FIG. 2E. When the flanges 249 are positioned in the lower cavity section 255B, the shoulder 257 overlies the flanges 249 and axially retains the flanges 249 of the lower end 222A of the screw 122 in the receptacle 251.

Within examples, the lower end 222A of the screw 122 and the receptacle 251 can have corresponding shapes and sizes that allow for the upper jaw 120 and the lower end 222A of the screw 122 to rotate relative to each other. For instance, the lower end 222A of the screw 122 and the receptacle 251 can have respectively circular shapes.

FIGS. 3A-3B depict an adapter 300 as another implementation of the adapter 100 shown and described with respect to FIG. 1, according to an example. In particular, FIG. 3A depicts a perspective view of the adapter 300 with the upper jaw 120 in a first position, and FIG. 3B depicts a perspective view of the adapter 300 with the upper jaw 120 in a second position, according to the example.

The adapter 300 shown in FIGS. 3A-3B is substantially similar or identical to the adapter 200 shown and described with respect to FIGS. 2A-2E, except the adapter 300 is configured such that the thumbscrew head 124 remains axially fixed relative to the clamp frame 110 in all positions of the upper jaw 120 relative to the clamp frame 110.

As shown in FIGS. 3A-3B, the adapter 300 includes the clamp frame 110. The clamp frame 110 includes the upper portion 112, the lower portion 114, and the bridge portion 116 between the upper portion 112 and the lower portion 114. As shown in FIGS. 3A-3B, the upper portion 112, the lower portion 114, and the bridge portion 116 are formed as a single, monolithic structure. This can help to enhance a structural strength and durability of the clamp frame 110, and/or simplify manufacturing of the clamp frame 110.

Additionally, as shown in FIGS. 3A-3B, the adapter 300 includes the lower jaw 118 and the upper jaw 120. In FIGS. 3A-3B, the lower jaw 118 and the lower portion 114 of the clamp frame 110 are formed a single, monolithic structure such that the lower portion 114 of the clamp frame 110 defines the lower jaw 118. However, in other implementations, the lower jaw 118 can be a separate component that is fixedly coupled to the lower portion 114 of the clamp frame 110.

As shown in FIGS. 3A-3B, the adapter 200 also includes the screw 122 that is threadably coupled to the upper portion 112 of the clamp frame 110. As described in further example below, in this example, the thumbscrew head 124 is coupled to the threaded section 247 of the screw 122 instead of the upper end of the screw 122. As described above, the lower end of the screw 122 can be rotatably coupled to the upper jaw 120 to allow the upper jaw 120 to remain rotationally fixed relative to the surgical instrument in the jaw space and the lower jaw 118 while the screw 122 is rotated to tighten the upper jaw 120 against the surgical instrument. Additionally, to help maintain the upper jaw 120 rotationally aligned with the lower jaw 118, the upper jaw 120 can be configured to abut against the bridge portion 116 of the clamp frame 110. In this arrangement, the upper jaw 120 can be rotationally aligned with the lower jaw 118 as the screw 122 moves the upper jaw 120 towards the lower jaw 118 and away from the lower jaw 118.

As shown in FIGS. 3A-3B, the upper jaw 120 is disposed in the frame cavity 234 of the clamp frame 110 and coupled to the screw 122. In this arrangement, the upper jaw 120 and the lower jaw 118 define the jaw space 236 for receiving the surgical instrument 201 between the upper jaw 120 and the lower jaw 118. As such, the jaw space 236 can be a portion of the frame cavity 234 that is between the upper jaw 120 and the lower jaw 118.

In FIGS. 3A-3B, the longitudinal axis 238 of the screw 122 extends between the upper end of the screw 122 and a lower end of the screw 122, and the longitudinal axis 238 of the screw 122 extends through the jaw space 236 defined by the upper jaw 120 and the lower jaw 118. As described above, this can decrease a risk of deflection of the upper jaw 120 when the surgical instrument 201 is in the jaw space 236 and a force is applied to the upper jaw 120 by the screw 122.

As noted above, in FIGS. 3A-3B, the thumbscrew head 124 is coupled to the threaded section 247 of the screw 122 instead of the upper end of the screw 122. For instance, the thumbscrew head 124 can include a lower portion 324A that is rotatably mounted to the upper portion 112 of the clamp frame 110. For instance, the lower portion 324A of the thumbscrew head 124 can be disposed within a retention channel 359 in the upper portion 112 of the clamp frame 110. The thumbscrew head 124 also includes an upper portion 324B that extends above the upper portion 112 of the clamp frame 110.

The lower portion 324A of the thumbscrew head 124 and the retention channel 359 can be configured such that (i) the lower portion 324A of the thumbscrew head 124 is rotatable within the retention channel 359, and (ii) the lower portion 324A of the thumbscrew head 124 is axially restrained within the retention channel 359. For instance, in the example shown in FIGS. 3A-3B, the lower portion 324A can include a neck portion that extends between the upper portion 324B and a disc portion, and the retention channel 359 can include a narrow constriction at an upper portion and an enlarged circular cavity below the narrow constriction. In this arrangement, the disc portion can rotate and be axially retained within the enlarged circular cavity of the retention channel 359, and the neck portion can extend through and rotate within the narrow constriction of the retention channel 359.

As shown in FIGS. 3A-3B, the upper portion 324B of the thumbscrew head 124 is above the upper portion 112 of the clamp frame 110. The screw 122 extends axially through the thumbscrew head 124 and is threadably coupled to the thumbscrew head 124. As shown in FIGS. 3A-3B, the thumbscrew head 124 extends through the thumbscrew head 124 such that at least a portion of the thumbscrew head 124 extends into the frame cavity 234. This provides for the lower end of the screw 122 to be coupled to the upper jaw 120.

In this arrangement, the thumbscrew head 124 is configured to rotate relative to the clamp frame 110 without moving axially (e.g., along a dimension that is parallel to the longitudinal axis 238). When the thumbscrew head 124 rotates in a first direction relative to the clamp frame 110, the thumbscrew head 124 drives the screw 122 in a first axial direction to move the upper jaw 120 towards the lower jaw 118. When the thumbscrew head 124 rotates in a second direction relative to the clamp frame 110, the thumbscrew head 124 drives the screw 122 in a second axial direction to move the upper jaw 120 away from the lower jaw 118.

FIG. 3A depicts the adapter 300 with the upper jaw 120 in the first position at which the upper jaw 120 nearer to the upper portion 112 of the clamp frame 110 than the lower portion 114 of the clamp frame 110 (e.g., the upper jaw 120 can be at the upper portion 112 of the clamp frame 110). FIG. 3B depicts the adapter 300 with the upper jaw in the second position at which the upper jaw 120 is nearer to the lower portion 114 of the clamp frame 110 than the upper portion 112 of the clamp frame 110 (e.g., the upper jaw 120 can be at the lower portion 114 of the clamp frame 110). As shown in FIGS. 3A-3B, the thumbscrew head 124 remains at the same axial position relative to the clamp frame 110 regardless of the position of the upper jaw 120. The implementation described above for FIGS. 3A-3B beneficially allows for the adapter 300 to have a smaller height compared to other implementations in which the thumbscrew head 124 is coupled to the upper end of the screw 122.

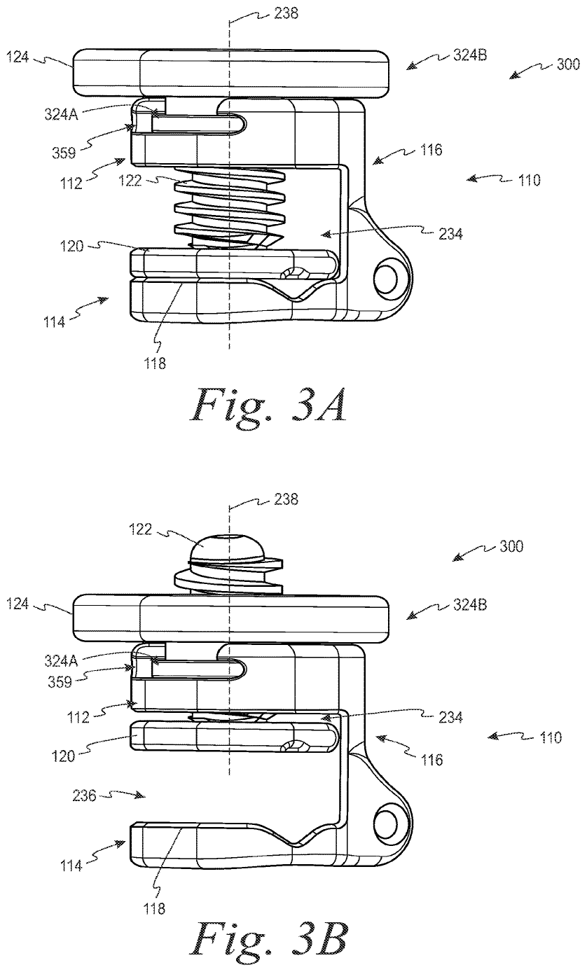

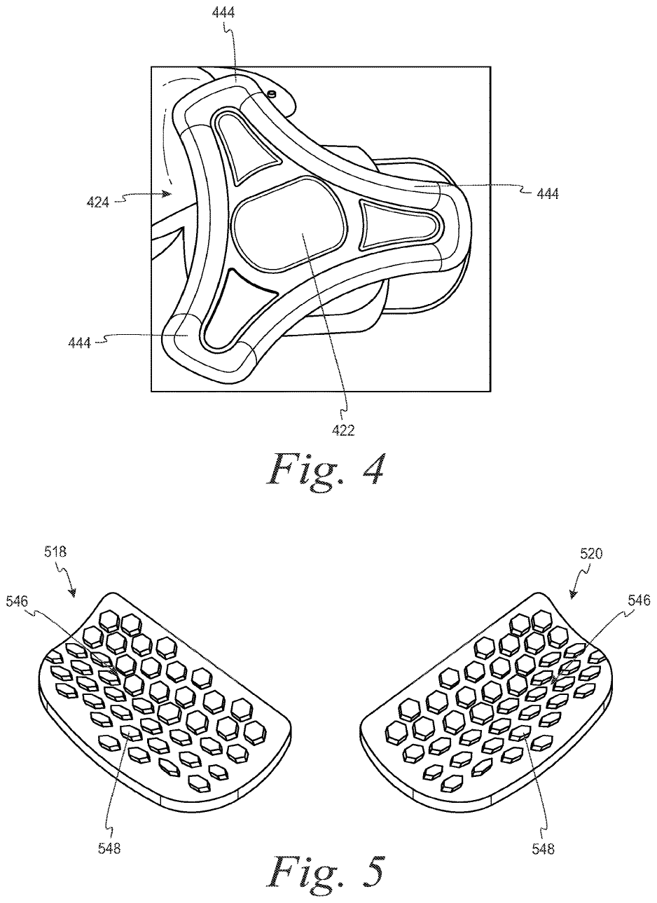

In the example shown in FIGS. 2A-3B, the thumbscrew head 124 has an elongated shape with two major portions extending from the screw 122. FIG. 4 depicts a thumbscrew head 424 as another implementation of the thumbscrew head 124 described above, according to another example. As shown in FIG. 4, the thumbscrew head 424 includes three lobes 444 extending in a plane that is orthogonal to the longitudinal axis 238 (shown in FIGS. 2A-2B) of the screw 122. This can help to provide improved mechanical advantage when using the thumbscrew head 424 to rotate the screw 122 as compared to the thumbscrew head 124 shown in FIGS. 2A-3B.

FIG. 5 depicts an upper jaw 520 and a lower jaw 518 as another implementation of the upper jaw 120 and the lower jaw 118 described above, according to an example. As shown in FIG. 5, the upper jaw 520 and the lower jaw 518 can each include a textured surface 546 that is configured to grip the surgical instrument 201. For instance, each textured surface 546 can include a plurality of grip protrusions 548. The textured surface 546 can help to increase friction between (i) the upper jaw 520 and the lower jaw 518, and (ii) the surgical instrument 201 positioned in the jaw space 236.

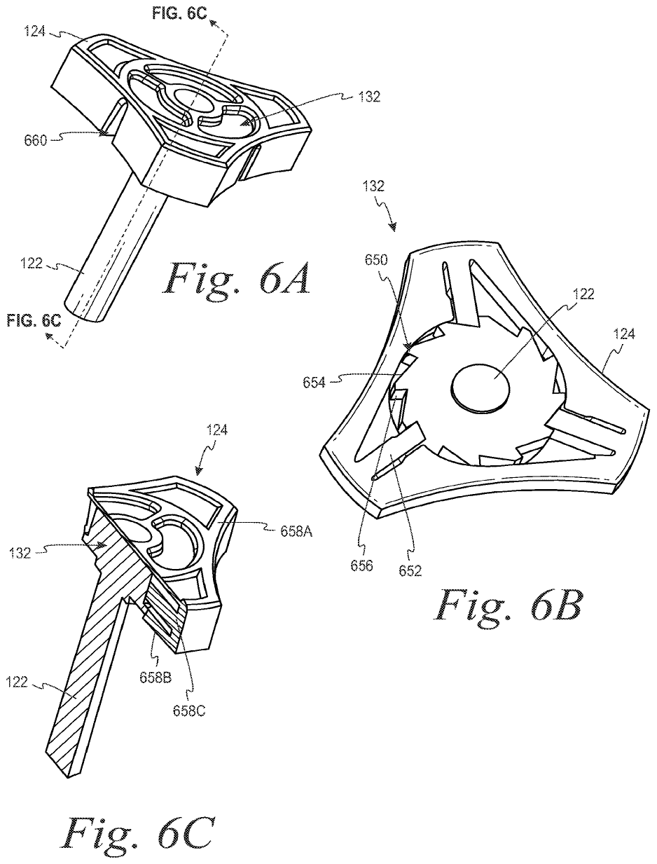

As described above, in some examples, the adapter 100, 200, 300 can include the torque limiter 132 to help achieve a more consistent and reliable force between the adapter 100, 200, 300 and the surgical instrument 201. FIGS. 6A-6C depict an implementation of the screw 122, the thumbscrew head 124, and the torque limiter 132, according to an example. In particular, FIG. 6A depicts a perspective view of an assembly of the screw 122, the thumbscrew head 124, and the torque limiter 132, FIG. 6B depicts a bottom view of the assembly shown in FIG. 6A, and FIG. 6C depicts a cross-sectional view of the assembly shown in FIG. 6A, according to the example.

As shown in FIGS. 6A-6C, the torque limiter 132 is configured to couple the thumbscrew head 124 to the screw 122. More particularly, the torque limiter 132 is configured to rotationally couple the thumbscrew head 124 to the screw 122 when a torque applied to the thumbscrew head 124 in a first rotational direction is less than the predetermined threshold amount of torque, and the torque limiter 132 is configured rotationally decouple the thumbscrew head 124 from the screw 122 when the torque applied to the thumbscrew head 124 in the first rotational direction is greater than the predetermined threshold amount of torque. Additionally, the torque limiter 132 is configured to rotationally couple the thumbscrew head 124 to the screw 122 when any torque is applied to the thumbscrew head 124 in a second rotational direction. In this arrangement, the torque limiter 132 is configured such that (i) rotating the thumbscrew in the first rotational direction with a torque that is less than the predetermined threshold amount of torque causes the upper jaw 120 to move towards the lower jaw 118 to decrease a size of the jaw space 236, and (ii) rotating the thumbscrew in the second rotational direction causes the upper jaw 120 to move away from the lower jaw 118 to increase the size of the jaw space 236.

In the example shown in FIGS. 6A-6C, the torque limiter 132 includes a plurality of ramps 650 arranged around a circumference of the screw 122, and a plurality of protrusions 652 extending from the thumbscrew head 124 and engaging the plurality of ramps 650 of on the screw 122. The ramps 650 and the plurality of protrusions 652 are configured such that: (i) when the torque applied to the thumbscrew head 124 in the first rotational direction is less than the predetermined threshold amount of torque, the protrusions 652 engage the ramps 650 and transmit the torque from the thumbscrew head 124 to the screw 122, and (ii) when the torque applied to the thumbscrew head 124 in the first rotational direction is greater than the predetermined threshold amount of torque, the protrusions 652 slip over and disengage from the ramps 650 to prevent transmission of the torque from the thumbscrew head 124 to the screw 122.

As shown in FIG. 6B, each ramp 650 can include an inclined surface 654 and a radial surface 656. Each radial surface 656 can extend in a direction that is substantially parallel to a radius of the screw 122. The ramps 650 and the protrusions 652 can be configured such that each protrusion 652 engages the radial surface 656 of a respective one of the ramps 650 when any amount of torque is applied to the thumbscrew head 124 in the second rotational direction.

Additionally, for each ramp 650, the inclined surface 654 can have a slope that defines, at least in part, the predetermined threshold amount of torque. Within examples, the predetermine threshold amount of torque can be further defined by a size and/or a shape of the protrusions 652 that engage the inclined surfaces 654 of the ramps 650. For instance, as shown in FIGS. 6A and 6C, the thumbscrew head 124 can include a top wall 658A, a bottom wall 658B, and a side wall 658C extending between the top wall 658A and the bottom wall 658B. The protrusions 652 can extend inwardly from the side wall 658C toward the screw 122. In some implementations, each protrusion 652 can include an end having a sloped surface that corresponds to a slope of the inclined surfaces 654. This can help to increase a surface area of engagement between the protrusions 652 and the ramps 650, which can relate to an amount of friction between the protrusions 652 and the ramps 650 and assist in defining the predetermined amount of torque. As examples, the predetermined amount of torque can be an amount between approximately 0.19 Nm and approximately 0.38 Nm.

As shown in FIG. 6A, in some examples, the side wall 658C of the thumbscrew head 124 can one or more notches 660 extending from the bottom wall 658B toward the top wall 658A. The one or more notches 660 can be configured to allow a first portion of the side wall 658C to flex outwardly away from a second portion of the side wall 658C to provide for coupling the thumbscrew head 124 to the screw 122. This can help to provide for easier assembly of the screw 122, the thumbscrew head 124, and the torque limiter 132.

In some examples, the torque limiter 132 can be configured to generate an audible signal responsive to the torque applied to the thumbscrew head 124 in the first rotational direction being greater than the predetermined threshold amount of torque. For example, the ramps 650 and the protrusions 652 can be configured such that the protrusions 652 generate a clicking sound when the protrusions 652 slide over the ramps 650 (e.g., when each protrusion 652 slides off a top of the inclined surface 654 and contacts a bottom of a next ramp 650). This can additionally or alternatively provide a tactile signal as well. By providing the audible signal and/or the tactile signal to the user when the torque applied to the thumbscrew head 124 in the first rotational direction being greater than the predetermined threshold amount of torque, the torque limiter 132 can be configured to notify the user when the screw 122 has been fully tightened and the surgical instrument is secured between the upper jaw 120 and the lower jaw 118.

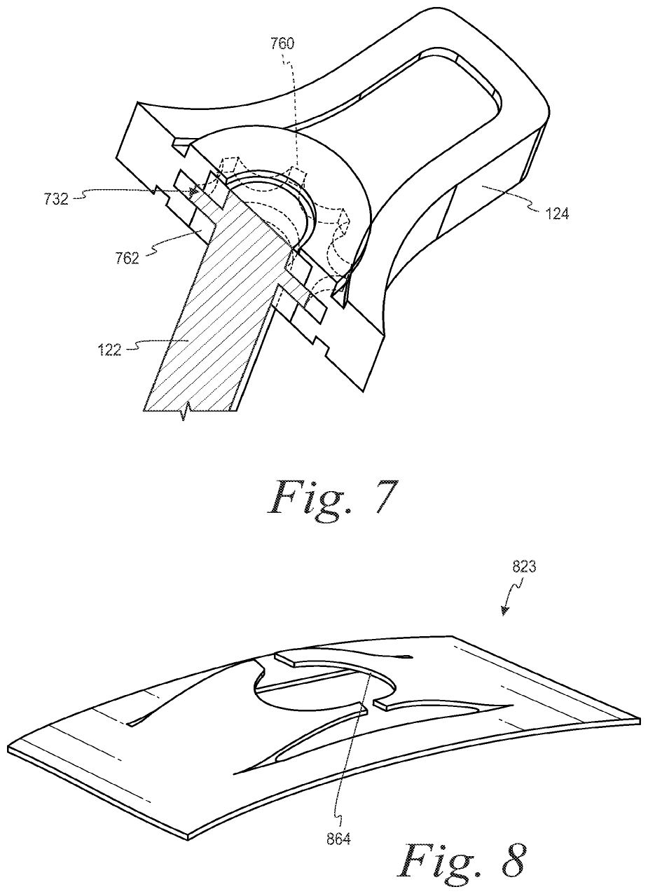

In another example, the torque limiter 132 can include a ball detent coupling between the thumbscrew head 124 and the screw 122. As one example, FIG. 7 depicts a cross-sectional view of an assembly of the screw 122, the thumbscrew head 124, and a torque limiter 732, according to another example. In this example, the thumbscrew head 124 is coupled to the screw 122 by a ball detent mechanism 760.

The ball detent mechanism 760 is configured to rotationally couple the thumbscrew head 124 to the screw 122 when a torque applied to the thumbscrew head 124 in a first rotational direction is less than the predetermined threshold amount of torque, and the ball detent mechanism 760 is configured rotationally decouple the thumbscrew head 124 from the screw 122 when the torque applied to the thumbscrew head 124 in the first rotational direction is greater than the predetermined threshold amount of torque. Additionally, the ball detent mechanism 760 is configured to rotationally couple the thumbscrew head 124 to the screw 122 when any torque is applied to the thumbscrew head 124 in a second rotational direction. In this arrangement, the ball detent mechanism 760 is configured such that (i) rotating the thumbscrew in the first rotational direction with a torque that is less than the predetermined threshold amount of torque causes the upper jaw 120 to move towards the lower jaw 118 to decrease a size of the jaw space 236, and (ii) rotating the thumbscrew in the second rotational direction causes the upper jaw 120 to move away from the lower jaw 118 to increase the size of the jaw space 236.

The ball detent mechanism 760 can include a plurality of balls that are biased towards a plurality of pockets (e.g., by springs). In one example, to limit torque in the first direction and not the second direction, the pockets can have an asymmetric geometry. In FIG. 6, the torque limiter 732 can also include a plurality of O-rings 762 to assist in coupling the screw 122, the thumbscrew head 124, and the torque limiter 732 to each other.

Additionally, in some examples, the ball detent mechanism 760 can be configured to generate the audible signal and/or a tactile signal responsive to the torque applied to the thumbscrew head 124 in the first rotational direction being greater than the predetermined threshold amount of torque.

As described above, the screw 122 includes an external thread that engages an internal thread of the clamp frame 110. In some implementations, the internal thread of the upper portion 112 of the clamp frame 110 can be integrally formed with the upper portion 112 of the clamp frame 110 as a single monolithic structure. In other implementations, the internal thread can be provided by a nut 123 that is coupled to the upper portion 112 of the clamp frame 110.

FIG. 8 depicts a nut 823 according to an example. In an implementation, the upper portion 112 of the clamp frame 110 shown in FIGS. 1, 2A, and 3A-3B can include the nut 823 having an internal thread 864 that is configured to engage an external thread of the screw 122. In the example shown in FIG. 8, the internal thread 864 is configured to deflect and disengage with the external thread of the screw 122 responsive to an axial force applied to the screw 122 in a direction from the upper jaw 120 towards the lower jaw 118. The internal thread 864 is configured to remain engaged with the external thread responsive to an axial force applied to the screw 122 in a direction from the lower jaw 118 towards the upper jaw 120. In this arrangement, the nut 823 can allow for a more rapid and efficient process of decreasing the jaw space 236 to couple the adapter 100, 200, 300 to the surgical instrument 201.

For instance, a process for coupling the adapter 100, 200, 300 to the surgical instrument 201 can include first positioning the surgical instrument 201 in the jaw space 236 while the upper jaw 120 is at a first distance from the lower jaw 118. After the surgical instrument 201 is positioned in the jaw space 236, the process can include applying a downward axial force to the screw 122 in the direction from the upper jaw 120 toward the lower jaw 118. This can cause the internal thread 864 of the nut 823 to disengage from the external thread of the screw 122, and allow the screw 122 and the upper jaw 120 to rapidly move toward the surgical instrument 201. Next, the downward axial force on the screw 122 can cease, which causes the internal thread 864 of the nut 823 to re-engage the external thread on the screw 122. After the internal thread 864 of the nut 823 re-engages the external thread of the screw 122, the thumbscrew head 124 can be rotated in the first direction to further decrease the jaw space 236 and increase a force applied to the surgical instrument 201 by the upper jaw 120 and the lower jaw 118. The nut 823 thus allows for more rapid coupling of the adapter 100, 200 to the surgical instrument 201 than would be possible if the only way to reduce the jaw space 236 was by rotating thumbscrew head 124 and the screw 122.

Nevertheless, the nut 123 can have an internal thread that is not deflectable in other examples. This can help to simplify manufacturing, simplify assembly, and/or reduce a cost of manufacture.

In the example implementation described above with respect to FIGS. 2A-3B, the longitudinal axis 238 of the screw 122 extends through the jaw space 236 defined by the upper jaw 120 and the lower jaw 118. That can decrease a risk of deflection of the upper jaw 120 when the surgical instrument 201 is in the jaw space 236 and a force is applied to the upper jaw 120 by the screw 122. As a result, the arrangement of the screw 122, the upper jaw 120, and the lower jaw 118 can address some problems associated with some adapters in which the screw 122 is offset laterally from the jaw space 236 (e.g., problems of undesirable deflection of the upper jaw 120 as described above).

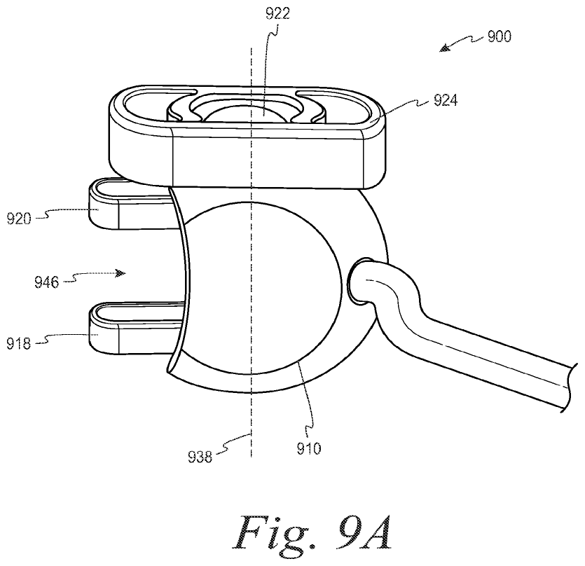

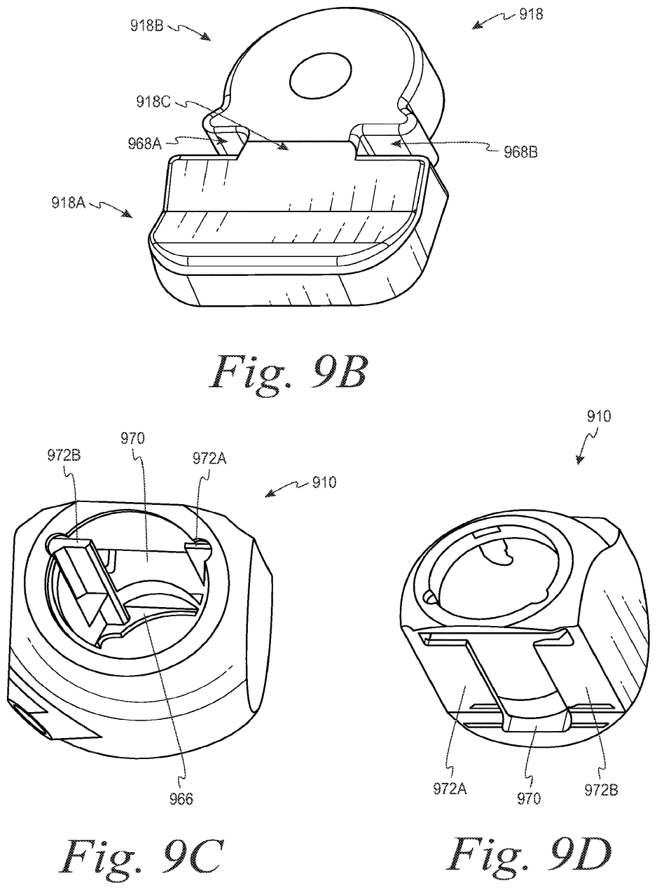

FIG. 9A depicts an adapter 900, according to another example. The adapter 900 is substantially similar to the adapter 100 described above, except (i) the adapter 900 includes an upper jaw 920 that is fixed relative to a clamp frame 910 and a lower jaw 918 that is movable relative to the clamp frame 910 and the upper jaw 920 by a screw 922, and (ii) a longitudinal axis 938 of the screw 922 is offset from a jaw space 946 between the upper jaw 920 and the lower jaw 918. As shown in FIG. 9A, the adapter 900 can also include a thumbscrew 924 coupled to the screw 922 as described above.

FIG. 9B depicts the lower jaw 918 for the adapter 900 shown in FIG. 9A and FIGS. 9C-9D depict the clamp frame 910 for the adapter 900 shown in FIG. 9A, according to an example. As shown in FIG. 9B, the lower jaw 918 can include (i) a clamp portion 918A that is configured to engage the surgical instrument 201 (shown in FIG. 2B), (ii) a screw-engagement portion 918B that is configured to couple the lower jaw 918 to the screw 922, and (iii) a neck portion 918C between the clamp portion 918A and the screw-engagement portion 918B. The neck portion 918C can have a width that is less than a width of the clamp portion 918A and a width of the screw-engagement portion 918B. In this arrangement, the clamp portion 918A, the screw-engagement portion 918B, and the neck portion 918C can define a first slot 968A on a first lateral side of the lower jaw 918 and a second slot 968B on a second lateral side of the lower jaw 918.

As shown in FIGS. 9C-9D, the clamp frame 910 can include an internal cavity 966 in which the screw 922 and the image guidance sensor 126 (shown in FIG. 1) can be disposed. The clamp frame 910 can include a channel 970, a first longitudinal flange 972A on a first lateral side of the channel 970, and a second longitudinal flange 972B on a second lateral side of the channel 970. The channel 970, the first longitudinal flange 972A, and the second longitudinal flange 972B can each have respective longitudinal axes that are parallel to the longitudinal axis 938 of the screw 922.

The lower jaw 918 can be coupled to the clamp frame 910 such that the screw-engagement portion 918B is in the internal cavity 966 of the clamp frame 910, the neck portion 918C extends through the channel 970 of the clamp frame 910, and the clamp portion 918A extends outwardly from the clamp frame 910. Additionally, the first longitudinal flange 972A of the clamp frame 910 can be received in the first slot 968A of the lower jaw 918, and the second longitudinal flange 972B of the clamp frame 910 can be received in the second slot 968B of the lower jaw 918. In this arrangement, an engagement between the first longitudinal flange 972A of the clamp frame 910 and the first slot 968A of the lower jaw 918, and an engagement of the second longitudinal flange 972B of the clamp frame 910 and the second slot 968B of the lower jaw 918 can mitigate (or prevent) deflection of the lower jaw 918 relative to the clamp frame 910 when the upper jaw 920 and the lower jaw 918 apply a force to the surgical instrument 201 in the jaw space 946.

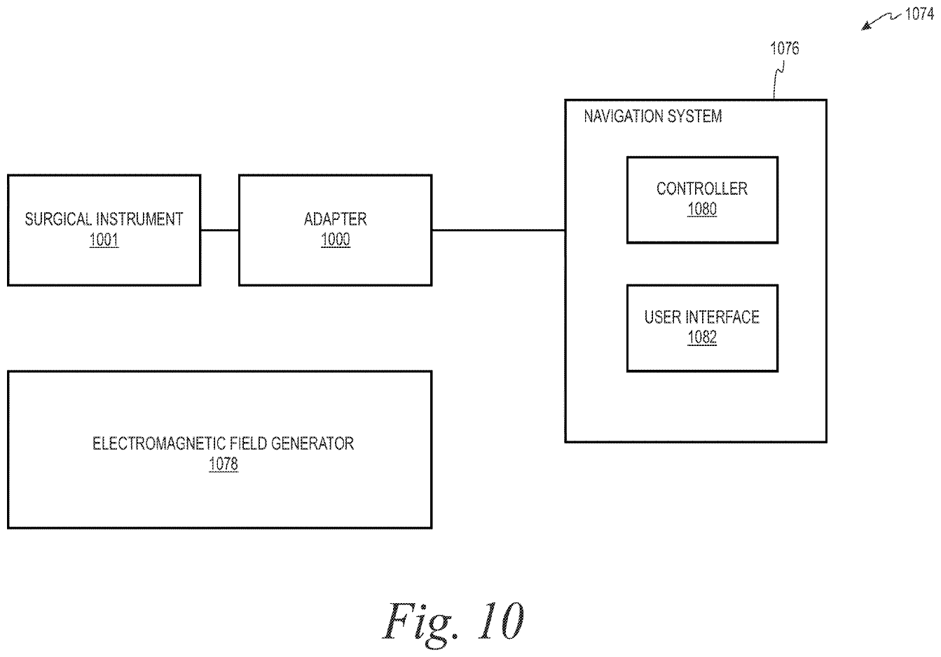

Referring now to FIG. 10, a simplified block diagram of an image guidance system 1074 is shown according to an example. As shown in FIG. 10, the image guidance system 1074 can include an adapter 1000, a surgical instrument 1001, a navigation system 1076, and/or an electromagnetic field generator 1078. The adapter 1000 can be an implementation of any of the adapters 100, 200, 300, 900 described above. As such, the adapter 1000 can include any feature described above, omit any feature described above, and/or be any combination of features described above for the adapters 100, 200, 300, 900.

The adapter 1000 is configured to couple to the surgical instrument 1001. For instance, the surgical instrument 1001 can include a shaft and the adapter 1000 can clamp onto to the shaft of the surgical instrument 1001. As examples, the surgical instrument 1001 can include one or more surgical instruments selected from a group consisting of: a microdebrider, an endoscope, a suction device, an irrigation device, a seeker device, and a frontal sinus forceps.

The adapter 1000 can be communicatively coupled to the navigation system 1076. The navigation system 1076 can include a controller 1080, which can be implemented using hardware, software, and/or firmware. For example, the controller 1080 can include one or more processors and a non-transitory computer-readable medium (e.g., volatile and/or non-volatile memory) that stores machine language instructions or other executable instructions. The instructions, when executed by the one or more processors, cause the navigation system 1076 to carry out various operations described herein. The controller 1080, thus, can receive data (including data indicated by the image guidance sensor 126 of the adapter 1000) and store the data in memory as well.

The processor(s) and/or the non-transitory computer-readable medium can be implemented in any number of physical devices/machines. For example, the controller 1080 can include one or more shared or dedicated general purpose computer systems/servers. Accordingly, principles and advantages of distributed processing, such as redundancy, replication, and the like, also can be implemented, as desired, to increase the robustness and performance of the devices and systems of the controller 1080.

The physical devices/machines can be implemented by the preparation of integrated circuits or by interconnecting an appropriate network of conventional component circuits, as is appreciated by those skilled in the electrical art(s). The physical devices/machines, for example, may include field programmable gate arrays (FPGA's), application-specific integrated circuits (ASIC's), digital signal processors (DSP's), etc. The physical devices/machines may reside on a wired or wireless network, e.g., LAN, WAN, Internet, cloud, near-field communications, etc., to communicate with each other and/or other systems, e.g., Internet/web resources.

The navigation system 1076 can additionally or alternatively include a user interface 1082 that can receive one or more inputs from a user and/or provide one or more outputs to the user. As examples, the user interface 1082 can include one or more buttons, one or more switches, one or more dials, one or more keypads, one or more touchscreens, one or more display devices, one or more indicator lights, one or more speakers, and/or one or more haptic output devices. The user interface 1082 is communicatively connected with the controller 1080.

The electromagnetic field generator 1078 can be configured to generate an electromagnetic field that can be sensed by the image guidance sensor 126 of the adapter 1000. In this arrangement, the electromagnetic field generator 1078 can generate the electromagnetic field at a surgical site. The image guidance sensor 126 of the adapter 1000 can sense the electromagnetic field generated by the electromagnetic field generator 1078 at the surgical site, and, based on the electromagnetic field sensed by the image guidance sensor 126, generate the signal that is indicative of the position of the image guidance sensor 126. The controller 1080 can receive signal from the image guidance sensor 126 of the adapter 1000 and cause the user interface 1082 to display an indication of a location and/or an orientation of the surgical instrument 1001 within the body of the patient (e.g., by displaying a position of the surgical instrument overlaid on the preoperative images). Thus, the surgeon performing the procedure is able to see where, in the body, the instrument is located even though the surgical site is not directly exposed to the eye.

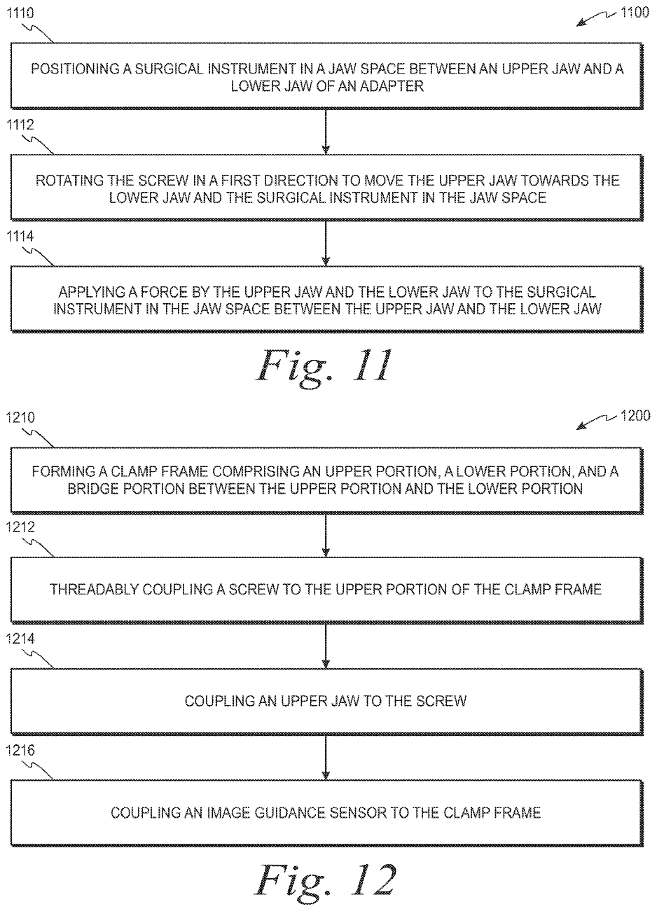

FIG. 11 depicts a flowchart for a process 1100 of coupling an adapter including an image guidance sensor to a surgical instrument, according to an example. As shown in FIG. 11, the process 1100 includes positioning a surgical instrument in a jaw space between an upper jaw and a lower jaw of an adapter at block 1110. The adapter includes a clamp frame, a screw, and an image guidance sensor. The clamp frame includes an upper portion, a lower portion, and a bridge portion between the upper portion and the lower portion. The clamp frame defines a frame cavity between the upper portion and the lower portion, and the lower jaw is at the lower portion of the clamp frame. The screw is threadably coupled to the upper portion of the clamp frame. The upper jaw is disposed in the frame cavity of the clamp frame and coupled to the screw. The upper jaw and the lower jaw define the jaw space for receiving the surgical instrument between the upper jaw and the lower jaw. The frame cavity of the clamp frame provides access to the jaw space. The screw is configured to move upper jaw relative to the lower jaw to adjust a size of the jaw space between the upper jaw and the lower jaw. The image guidance sensor that is configured to generate a signal that is indicative of a position of the sensor responsive to an electromagnetic field generated by a field generator.

As shown in FIG. 11, the process 1100 also includes rotating the screw in a first direction to move the upper jaw towards the lower jaw and the surgical instrument in the jaw space at block 1112. Additionally, the process 1100 includes applying a force by the upper jaw and the lower jaw to the surgical instrument in the jaw space between the upper jaw and the lower jaw at block 1114.

FIG. 12 depicts a flowchart for a process 1200 of making an adapter including an image guidance sensor to a surgical instrument, according to an example. As shown in FIG. 12, the process 1200 includes forming a clamp frame comprising an upper portion, a lower portion, and a bridge portion between the upper portion and the lower portion at block 1210. The clamp frame defines a frame cavity between the upper portion and the lower portion. The lower portion of the clamp frame defines a lower jaw.

The process 1200 also includes threadably coupling a screw to the upper portion of the clamp frame at block 1212. Additionally, the process 1200 includes coupling an upper jaw to the screw at block 1214. The upper jaw is disposed in the frame cavity of the clamp frame. The upper jaw and the lower jaw define a jaw space for receiving the surgical instrument between the upper jaw and the lower jaw. The frame cavity of the clamp frame provides access to the jaw space. The screw is configured to move upper jaw relative to the lower jaw to adjust a size of the jaw space between the upper jaw and the lower jaw.

The process 1200 further includes coupling an image guidance sensor to the clamp frame at block 1216. The image guidance sensor is configured to generate a signal that is indicative of a position of the sensor responsive to an electromagnetic field generated by a field generator.

The description of the different advantageous arrangements has been presented for purposes of illustration and description, and is not intended to be exhaustive or limited to the embodiments in the form disclosed. Many modifications and variations will be apparent to those of ordinary skill in the art. Further, different advantageous embodiments may provide different advantages as compared to other advantageous embodiments. The implementation or implementations selected are chosen and described in order to best explain the principles of the embodiments, the practical application, and to enable others of ordinary skill in the art to understand the disclosure for various embodiments with various modifications as are suited to the particular use contemplated.

1. An adapter for coupling an image guidance sensor to a surgical instrument, comprising:
a clamp frame comprising an upper portion, a lower portion, and a bridge portion between the upper portion and the lower portion, wherein the clamp frame defines a frame cavity between the upper portion and the lower portion, wherein the lower portion of the clamp frame defines a lower jaw;
a screw that is threadably coupled to the upper portion of the clamp frame; and
an upper jaw that is disposed in the frame cavity of the clamp frame and coupled to the screw, wherein the upper jaw and the lower jaw define a jaw space for receiving the surgical instrument between the upper jaw and the lower jaw, wherein the frame cavity of the clamp frame provides access to the jaw space, wherein the screw is configured to move upper jaw relative to the lower jaw to adjust a size of the jaw space between the upper jaw and the lower jaw; and
an image guidance sensor that is configured to generate a signal that is indicative of a position of the image guidance sensor responsive to an electromagnetic field generated by a field generator.

2. The adapter of claim 1, wherein the screw has a longitudinal axis extending between an upper end of the screw and a lower end of the screw, and
wherein the longitudinal axis of the screw extends through the jaw space defined by the upper jaw and the lower jaw.

3. The adapter of claim 2, wherein the lower end of the screw is rotatably coupled to the upper jaw.

4. The adapter of claim 3, wherein the lower end of the screw comprises a plurality of resilient fingers that are separated by a plurality of axial slots,
wherein the resilient fingers are configured to flex radially inwardly, and
wherein the upper jaw comprises a receptacle that is configured to receive and axially retain the lower end of the screw.

5. The adapter of claim 1, wherein the upper jaw and the lower jaw each comprise a textured surface that is configured to grip the surgical instrument, wherein each textured surface comprises a plurality of grip protrusions.

6. The adapter of claim 1, wherein the upper jaw comprises a first recess and the lower jaw comprises a second recess that is opposite the first recess of the upper jaw, and
wherein the first recess has a center axis between a first lateral side of the upper jaw and a second lateral side of the upper jaw,
wherein the second recess has a center axis between a first lateral side of the lower jaw and a second lateral side of the lower jaw, and
wherein the center axis of the first recess is parallel to the center axis of the second recess.

7. The adapter of claim 6, wherein a shape of the first recess mirrors a shape of the second recess.

8. The adapter of claim 6, wherein the center axis of the first recess and the center axis of the second recess are orthogonal to a longitudinal axis of the screw.

9. (canceled)

10. The adapter of claim 1, further comprising a thumbscrew head coupled to an upper end of the screw, wherein the thumbscrew head is rotatable relative to the clamp frame to move the screw and the upper jaw relative to the clamp frame and the lower jaw.

11. (canceled)

12. The adapter of claim 10, further comprising a torque limiter configured to couple the thumbscrew head to the screw,
wherein the torque limiter is configured to rotationally couple the thumbscrew head to the screw when a torque applied to the thumbscrew head in a first rotational direction is less than a predetermined threshold amount of torque, and
wherein the torque limiter is configured rotationally decouple the thumbscrew head from the screw when the torque applied to the thumbscrew head in the first rotational direction is greater than the predetermined threshold amount of torque.

13. The adapter of claim 12, wherein the torque limiter is configured to rotationally couple the thumbscrew head to the screw when any torque is applied to the thumbscrew head in a second rotational direction.

14. The adapter of claim 13, wherein the torque limiter is configured such that (i) rotating the thumbscrew head in the first rotational direction with a torque that is less than the predetermined threshold amount of torque causes the upper jaw to move towards the lower jaw, and (ii) rotating the thumbscrew head in the second rotational direction causes the upper jaw to move away from the lower jaw.

15. The adapter of claim 13, wherein the torque limiter comprises:
a plurality of ramps arranged around a circumference of the screw; and
a plurality of protrusions extending from the thumbscrew head and engaging the plurality of ramps of on the screw, and
wherein the plurality of ramps and the plurality of protrusions are configured such that:
(i) when the torque applied to the thumbscrew head in the first rotational direction is less than the predetermined threshold amount of torque, the plurality of protrusions engage the plurality of ramps and transmit the torque from the thumbscrew head to the screw, and
(ii) when the torque applied to the thumbscrew head in the first rotational direction is greater than the predetermined threshold amount of torque, the plurality of protrusions slip over and disengage from the plurality of ramps to prevent transmission of the torque from the thumbscrew head to the screw.

16. The adapter of claim 15, wherein each ramp comprises an inclined surface and a radial surface,
wherein each radial surface extends in a direction that is substantially parallel to a radius of the screw, and
wherein the plurality of ramps and the plurality of protrusions are configured such that each protrusion engages the radial surface of a respective one of the plurality of ramps when any torque is applied to the thumbscrew head in the second rotational direction.

17. The adapter of claim 16, wherein the thumbscrew head comprises a top wall, a bottom wall, and a side wall extending between the top wall and the bottom wall,
wherein the plurality of protrusions extend inwardly from the side wall toward the screw, and
wherein the side wall comprises one or more notches extending from the bottom wall toward the top wall, and
wherein the one or more notches are configured to allow a first portion of the side wall to flex outwardly away from a second portion of the side wall to provide for coupling the thumbscrew head to the screw.

18. The adapter of claim 13, wherein the torque limiter is configured to generate an audible signal responsive to the torque applied to the thumbscrew head in the first rotational direction being greater than the predetermined threshold amount of torque.

19. The adapter of claim 18, wherein the torque limiter comprises a ball detent coupling between the thumbscrew head and the screw.

20. The adapter of claim 1, wherein the upper portion of the clamp frame comprises a nut having an internal thread that is configured to engage an external thread of the screw, and
wherein the internal thread is configured to deflect and disengage with the external thread of the screw responsive to an axial force applied to the screw in a direction from the upper jaw towards the lower jaw.

21. The adapter of claim 20, wherein the internal thread is configured to remain engaged with the external thread responsive to an axial force applied to the screw in a direction from the lower jaw towards the upper jaw.

22. The adapter of claim 1, further comprising a thumbscrew head coupled to a threaded section of the screw, wherein the thumbscrew head is rotatable relative to the clamp frame to move the screw and the upper jaw relative to the clamp frame and the lower jaw,
wherein the thumbscrew head comprises a lower portion that is rotatably mounted to the upper portion of the clamp frame,
wherein the lower portion of the thumbscrew head is disposed within a retention channel in the upper portion of the clamp frame, and
wherein the thumbscrew head comprises an upper portion that extends above the upper portion of the clamp frame.

23-30. (canceled)
